(19) United States
(12) Patent Application Publication
(10) Pub. No.: US 2015/0196391 A1
(43) Pub. Date: Jul. 16, 2015

(54) Tray for Loading a Medical Device Including a Temperature Measuring and Indicating Device

(71) Applicant: Medtronic, Inc., Minneapolis, MN (US)

(72) Inventor: Joshua Dwork, Santa Rosa, CA (US)

(73) Assignee: Medtronic, Inc., Minneapolis, MN (US)

(21) Appl. No.: 14/155,772

(22) Filed: Jan. 15, 2014

(51) Int. Cl. A61F 2/24

(52) U.S. Cl. CPC A61F 2/2427

(57) ABSTRACT

A tray for loading a medical device on a catheter assembly includes a reservoir and a temperature sensing and indicating device disposed in the reservoir. The reservoir is defined by a bottom surface, a first wall, a second wall, a third wall, and a fourth wall, and includes an open top opposite the bottom surface. The reservoir configured to receive a liquid such that a medical device may be loaded onto a catheter assembly while submerged in the liquid. The temperature sensing and indicating device senses the temperature of the liquid in the reservoir and indicates to the user when the liquid is at a desired temperature for loading the medical device onto the catheter assembly.

### FIELD OF THE INVENTION

The present invention relates to catheter assemblies, loading trays, and methods of loading a catheter assembly. More specifically, the present invention relates to loading trays including a temperature measuring and indicating device that informs the user when a liquid in the tray is at the proper temperature for loading a medical device on a catheter assembly.

### BACKGROUND

Heart valves, such as the mitral, tricuspid, aortic, and pulmonary valves, are sometimes damaged by disease or by aging, resulting in problems with the proper functioning of the valve. Heart valve problems generally take one of two forms: stenosis in which a valve does not open completely or the opening is too small, resulting in restricted blood flow; or insufficiency in which blood leaks backward across a valve when it should be closed.

Heart valve replacement has become a routine surgical procedure for patients suffering from valve regurgitation or stenotic calcification of the leaflets. Conventionally, the vast majority of valve replacements entail full stenotomy in placing the patient on cardiopulmonary bypass. Traditional open surgery inflicts significant patient trauma and discomfort, requires extensive recuperation times, and may result in life-threatening complications.

To address these concerns, within the last decade, efforts have been made to perform cardiac valve replacements using minimally-invasive techniques. In these methods, laparoscopic instruments are employed to make small openings through the patient's ribs to provide access to the heart. While considerable effort has been devoted to such techniques, widespread acceptance has been limited by the clinician's ability to access only certain regions of the heart using laparoscopic instruments.

Still other efforts have been focused upon percutaneous transcatheter (or transluminal) delivery of replacement cardiac valves to solve the problems presented by traditional open surgery and minimally-invasive surgical methods. In such methods, a valve prosthesis is compacted for delivery in a catheter and then advanced, for example through an opening in the femoral artery and through the descending aorta to the heart, where the prosthesis is then deployed in the valve annulus (e.g., the aortic valve annulus).

Valve prostheses are generally formed by attaching a bioprosthetic valve to a frame made of a wire or a network of wires, often referred to as a stent or stent frame. Such a valve prosthesis can be contracted radially to introduce the valve prosthesis into the body of the patient percutaneously through a catheter. The valve prosthesis can be deployed by radially expanding it once positioned at the desired target site. The valve prosthesis is mounted onto a distal tip of the catheter assembly prior to delivery to the target location where the valve prosthesis is expanded into place.

To prepare such a valve prosthesis for implantation, the valve prosthesis can be initially provided in an expanded or uncrimped condition, then crimped or compressed around the distal tip of the catheter assembly until the valve prosthesis is as close to the diameter of the distal tip as possible. Various methods and devices are available for crimping the valve prosthesis onto the catheter's distal tip, which may include hand-held devices or tabletop devices, for example. These crimping devices can initially provide an opening that is large enough to accommodate a valve prosthesis in its expanded condition and be positioned over a desired section of a distal tip of the catheter assembly. The valve prosthesis can then be compressed by reconfiguring the opening of the crimping device to uniformly decrease the size of the opening until the valve is compressed to the desired size. Due to the bioprosthetic valve, the valve prosthesis often is not shipped loaded into the delivery catheter. Instead, many transcatheter valve prostheses must be loaded into the catheter assembly by hand at the treatment facility (e.g., operating room) immediately prior to performance of the procedure.

The frames of many transcatheter valve prostheses are formed from a nickel-titanium alloy such as nitinol or other shape memory or self-expanding material. When loading the transcatheter valve into the delivery catheter, high loading forces may cause damage to the frame. Reducing these loading stresses by softening the frame material is desirable. Lowering the temperature of a nitinol frame to approximately 0° F.-8° C. softens the nitinol material of the frame such that loading forces on the frame are reduced, thereby reducing the potential of damage to the valve prosthesis during loading into the delivery catheter. Accordingly, there is a need for a device that exposes the frame to a reduced temperature and informs the user that the environment is at the desired temperature such that the prosthetic valve may be loaded into the catheter assembly.

### SUMMARY OF THE INVENTION

Embodiments hereof relate to a tray for loading a medical device on a catheter assembly. The tray includes a reservoir having a generally open top configured to receive a liquid. The medical device is loaded onto the catheter assembly while being at least partially submerged in the liquid. The tray further includes a temperature sensor disposed in the reservoir for measuring the temperature of the liquid and indicating when the liquid is at a desired temperature for loading the medical device on the catheter assembly.

Embodiments hereof also relate to a method of loading a medical device on a catheter assembly. The method includes the steps of filling a reservoir in a loading tray with a fluid, adding a temperature modifying agent to the fluid, waiting for a temperature sensor disposed in the reservoir to indicate that the fluid is at a desired temperature. The medical device is loaded on the distal tip of the catheter assembly while the medical device and the distal tip are submerged in the reservoir after the fluid is at the desired temperature.

### BRIEF DESCRIPTION OF DRAWINGS

FIG. 1 is schematic diagram of a catheter assembly for delivering a transcatheter prosthetic heart valve to an implantation site, with the transcatheter heart valve prosthesis loaded in the catheter assembly.

FIG. 2 is an exploded perspective view of a catheter assembly and a loading tray with a temperature sensing and indicating device disposed in a reservoir of the loading tray.

FIG. 3 is a schematic illustration of an embodiment of a mechanism for coupling the temperature sensing and indicating device to the loading tray.

FIG. 4 is another embodiment of a temperature sensing and indicating device and mechanism for coupling the device to the loading tray.

FIG. 5 is a perspective view of the loading tray of FIG. 2 with a catheter assembly seated therein for loading.

FIG. 5 is a perspective view of the loading tray of FIG. 2 with a catheter assembly seated therein and a reservoir filled with a fluid for loading.

FIG. 7 is an exploded perspective view of a catheter assembly and a loading tray with a visualization device and a temperature sensing and indicating device disposed in the loading tray.

FIG. 8 is a top view of the loading tray of FIG. 7.

### DETAILED DESCRIPTION

Specific embodiments of the present invention are now described with reference to the figures, wherein like reference numbers indicate identical or functionally similar elements. Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” is used in the following description with reference to one or more stent structures of the prostheses hereof and is intended to convey that the structures are shaped or formed from a material that can be provided with a mechanical memory to return the structure from a compressed or constricted delivery configuration to an expanded deployed configuration. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in embodiments hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

The following detailed description is merely exemplary in nature and is not intended to limit the invention or the application and uses of the invention. Although the description of the invention is in the context of loading a heart valve prosthesis onto a catheter assembly, the devices and methods described herein can also be used for loading other devices onto catheter assemblies. Furthermore, there is no intention to be bound by any expressed or implied theory presented in the preceding technical field, background, brief summary or the following detailed description.

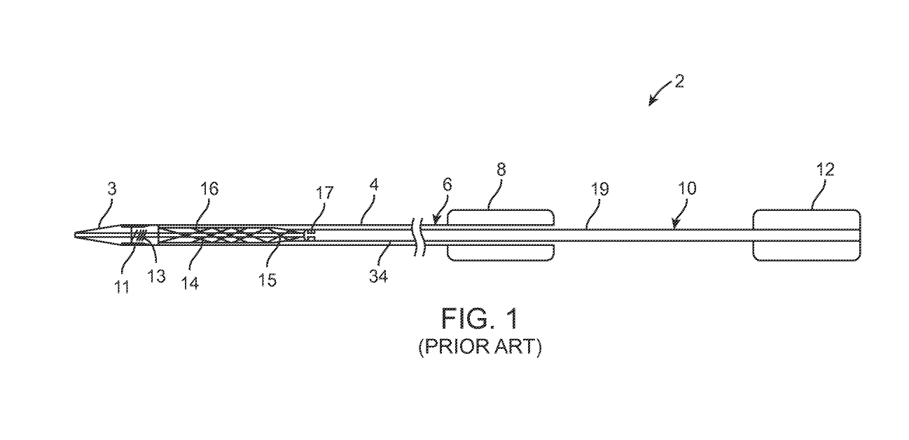

FIG. 1 is a diagram illustrating an example of a catheter assembly 2 for delivering a transcatheter heart valve prosthesis 14 to an implantation site. As noted above, heart valve prosthesis 14 includes a frame 16 and a prosthetic valve (note shown). In the illustrated example, catheter assembly 2 includes a shaft assembly 10 and a sheath assembly 6. The shaft assembly 10 includes a handle 12, a carrier shaft 19, a connector shaft 15, a distal tip assembly 3, a distal coupling structure 13, and a sleeve 11. The connector shaft 15 interconnects the carrier shaft 19 and the distal tip assembly 3, and in some constructions has a reduced-sized diameter to permit placement of a prosthetic heart valve 14 over the connector shaft 15. The distal tip assembly 3 is disposed at the distal end of the shaft assembly 10. Though not shown in FIG. 1, a guide wire lumen can be formed through shafts 15 and 19.

Carrier shaft 19 is sized to be slidably received within the sheath assembly 6, and is configured in the illustrated exampled for releasable coupling with the heart valve prosthesis 14. The carrier shaft 19 forms or includes a coupling device 17. The coupling device 17 is configured to selectively retain a proximal portion of the heart valve prosthesis 14. The coupling device 17 is configured to releasably mount the heart valve prosthesis 14 to the shaft assembly 10 when the heart valve prosthesis 14 is forced to a collapsed state within the sheath assembly 6. The sheath assembly 6 is configured to permit deployment of the heart valve prosthesis 14 from the loaded state shown in FIG. 1. The catheter assembly 2 is configured to transition from the loaded state in which the sheath assembly 6 encompasses the heart valve prosthesis 14 to a deployed state in which the sheath assembly 6 is withdrawn from the heart valve prosthesis 14.

The catheter assembly 2 shown in FIG. 1 is merely an example of delivery system that can be used to deliver a heart valve prosthesis transluminally to a desired treatment site. Further description of catheter assembly 2 can be found in U.S. Patent Application Publication No. 2011/0264198, the entirety of which is incorporated by reference herein.

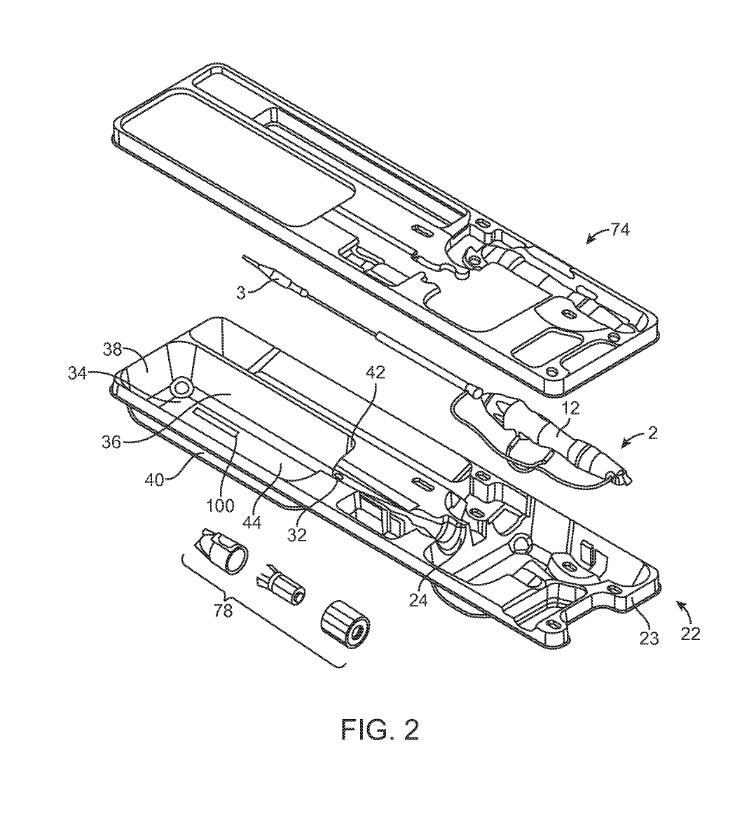

As discussed above, loading of heart valve prosthesis 14 onto catheter assembly 2 is generally performed in a liquid solution disposed in a reservoir of a loading tray. FIG. 2 shows an embodiment of a loading tray 22 including a temperature sensing and indicating device 100. Other embodiments of the loading tray and visualization device are possible. Modifications can be made to the embodiments described herein without departing from the spirit and scope of the present invention. Therefore, the following detailed description is not meant to be limiting. Further, the systems and methods described below can be implemented in many different embodiments of hardware. Any actual hardware described is not meant to be limiting. The operation and behavior of the systems and methods presented are described with the understanding that modifications and variations of the embodiments are possible given the level of detail presented.

In the embodiment shown in FIG. 2, loading tray 22 is configured to be used with a catheter assembly 2 that may be similar to the catheter assembly 2 shown in FIG. 1. As described above, catheter assembly 2 generally includes a handle 12 located at a proximal end of catheter assembly 2, a distal tip assembly 3, and a sheath assembly 6 between distal tip assembly 3 and handle assembly 12. It is understood that catheter assembly 2 is merely an exemplary embodiment of a catheter assembly that can be used in conjunction with the devices described herein. Similarly, loading tray 22, described in detail below, is merely an exemplary embodiment of a loading tray that can be used in conjunction with the temperature sensing and indicating device described herein. The present invention is not limited to the temperature sensing and indicating devices that can be used with loading trays and catheter assemblies as the one described herein. The temperature sensing and indicating devices described herein can be used with loading trays having different configurations of reservoirs and receptacles, and with catheter assemblies having different types of handle assemblies, sheath assemblies, and distal tip assemblies.

Loading tray 22 will be described briefly herein in conjunction with the temperature sensing and indicating device described herein. Loading tray 22, however, can be any conventional loading tray adapted to include the visualization devices described herein. For example, and not by way of limitation, loading tray 22 can be the loading tray described in U.S. Patent Application Publication No. 2012/0103840, which is incorporated in its entirety by reference herein. Briefly, loading tray 22 is made of a tray body 23 defining a handle assembly receptacle 24 for seating handle assembly 12 of catheter assembly 2, an elongate delivery shaft receptacle 28 for seating sheath assembly 6 of catheter assembly 2, and a reservoir 34 for holding a fluid (not shown in FIG. 2). Tray body 23 can be made of various polymer or composite materials including, for example, Polyethylene Terephthalate Glycol (PETG). Tray body 23 can be molded and have a thickness of approximately 1.0-1.4 mm. The present invention, however, is not limited to polymer materials and can include other suitable materials, for example, stainless steel. A top surface 46 of tray body 23 generally defines the uppermost horizontal plane of loading tray 22.

Reservoir 34 has a bottom surface 44 that is below a portion of delivery shaft receptacle 28 that is contiguous with reservoir 34. When reservoir 34 is filled with a fluid and handle assembly 12 is seated in the handle assembly receptacle 24, distal tip assembly 3 is submerged in the fluid in reservoir 34.

In the present embodiment, reservoir 34 is defined by a right wall 36, a back wall 38, a left wall 40, and a front wall 42 that extend downward from top surface 46 to horizontal bottom surface 44 to form a rectangular recess. The depth of the reservoir 34 may vary depending upon the depth necessary to load a medical device on distal tip assembly 3 while submerged in the fluid in reservoir 34. For example, when loading tray 22 is used to load a heart valve prosthesis on catheter assembly 2, the depth of reservoir 34 can be approximately 62-68 mm. Although reservoir 34 is rectangular in the illustrated embodiment, the present invention includes a tray that defines reservoirs having other shapes, for example, hemispheres, squares, and cylinders.

In an embodiment, loading tray 22 may also include a cover 74, as shown in FIG. 2. Further, a crimping device 78 can be stored in loading tray 22 for delivery. Similarly, the loading tray may be modified to accommodate any of the temperature sensing and indicating devices described in the embodiments below such that the temperature sensing and indicating devices may be shipped with the loading tray, crimping device, catheter, medical device, and/or other devices associated with the procedure for which catheter is intended. Other features of loading tray 22 shown in FIG. 2 are not described herein, but are explained in U.S. Patent Application Publication No. 2012/0103840, which is incorporated in its entirety by reference herein.

In the embodiment shown in FIG. 2, a temperature sensing and indicating device 100 is disposed within reservoir 34 of loading tray 22. Temperature sensing and indicating device 100 senses or measures the temperature of a liquid disposed in reservoir 34 and indicates the temperature such that a user can determine the appropriate time to load the heart valve prosthesis 14 into the catheter 2. In the embodiment of FIG. 2, temperature sensing and indicating device 100 is coupled to bottom surface 44 of reservoir 34. However, those skilled in the art would recognize that temperature sensing and indicating device 100 may be disposed anywhere such that an accurate measurement of temperature of the fluid in reservoir 34 may be made and displayed to the user. Further, the temperature sensing portion and the indicating or display portion may be separated.

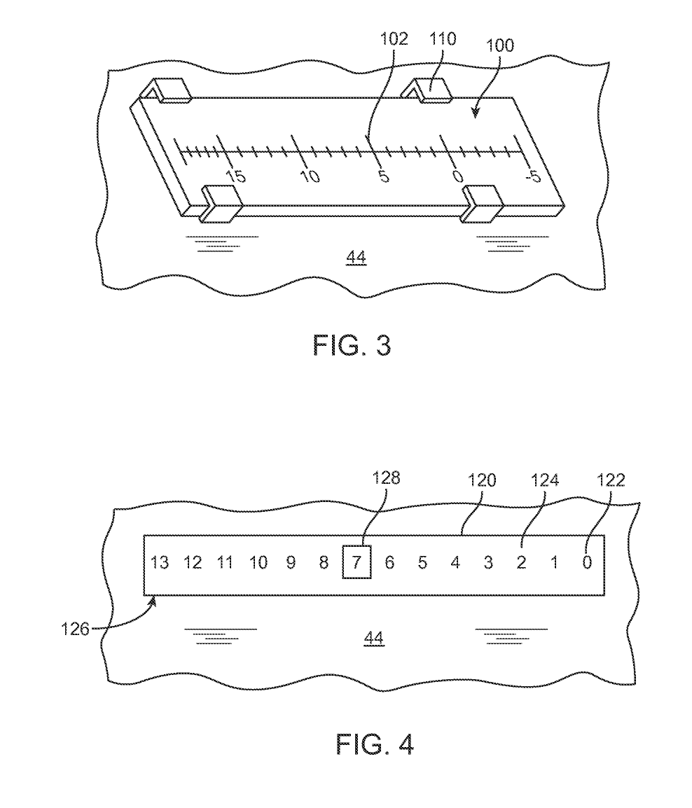

FIG. 3 schematically shows a portion or reservoir 34 with temperature sensing and indicating device 100 coupled to bottom surface 44 thereof. In the embodiment of FIG. 3, tabs or clips 110 extend from bottom surface 44 of reservoir 24 and temperature sensing and indicating device 100 is snap-fit under tabs 110 to hold it in place. Further, as seen in FIG. 3, temperature sensing and indicating device 100 includes markings 102 such that a user can reading the temperature measured by temperature sensing and indicating device 100. In the embodiment of FIG. 3, markings 102 are in the form of gradations marked with temperatures in the desired temperature range.

FIG. 4 schematically shows another embodiment of a temperature sensing and indicating device 120 coupled to bottom surface 44 of reservoir 34. In the embodiment of FIG. 4, temperature sensing and indicating device 120 is adhesively coupled to bottom surface 44. Accordingly, a bottom surface 124 of temperature sensing and indicating device 120 includes an adhesive such that it sticks to bottom surface 44 of reservoir 34. Temperature sensing and indicating device 120 may be adhesively coupled to bottom surface 44 prior to shipment or it may be adhesively coupled to bottom surface 44 by the user. Further, although FIG. 4 shows temperature sensing and indicating device 120 adhesively coupled to bottom surface 44, those skilled in the art would recognize that temperature sensing and indicating device 120 may be adhesively coupled to any surface of reservoir 34 provided a temperature reading of a fluid disposed in reservoir 34 may be taken. The adhesive used may be a pressure sensitive adhesive. A top surface 124 of temperature sensing and indicating device 120 includes markings 122 indicating the temperature reading. In the embodiment of FIG. 4, markings 122 are individual numbers indicating the temperature; wherein the number is highlighted 128 to indicate the temperature reading. The highlight 128 can be a box or other polygon around the number, as shown in FIG. 4, a pre-selected color, or other mechanism to indicate the temperature reading.

As would be apparent to those skilled in the art, other temperature sensing and indicating devices may be utilized provided that they can indicate to a user when a liquid in reservoir 34 has reached a desired temperature or temperature range, as described below. For example, one or more temperature thermocouples and a temperature sensing or indicating digital or analog display device coupled to the one or more thermocouples may be utilized. The one or more thermocouples may be positioned in or coupled to the reservoir. Further, instead of indicating the actual temperature, the temperature sensing and indicating device may merely indicate whether or not the liquid is in the desired temperature range. For example, and not by way of limitation, the temperature sensing and indicating device may show a red display when the temperature is not in the desired range and a green display when the temperature is in the desired range. Other indications, such as “do not load” and “load” may be used to indicate that the liquid is in the desired temperature range, as would be known to those skilled in the art. In another embodiment, an audible alarm indicating that the liquid is in the desired temperature range may be used. One or more visual or audible signals or alarms may be used in combination or separately to indicate or provide an alert when the temperature is in or out of the desired range.

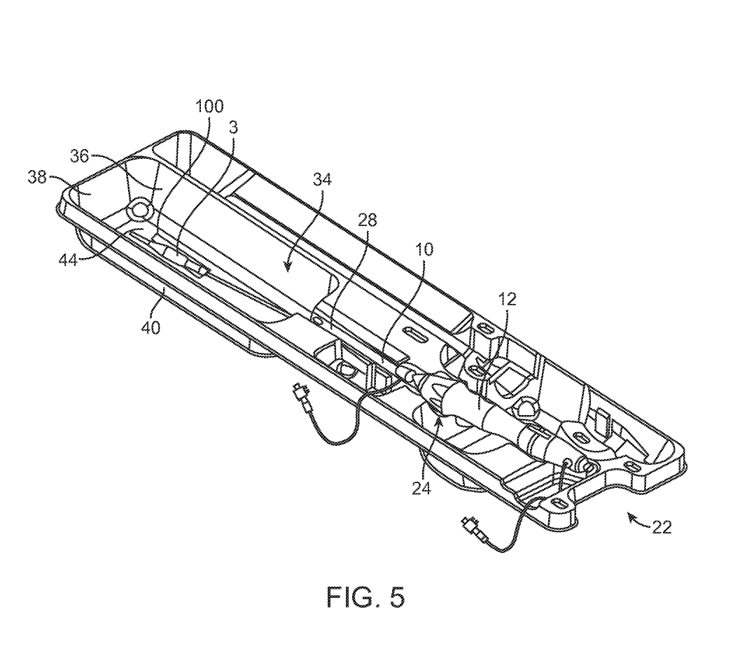

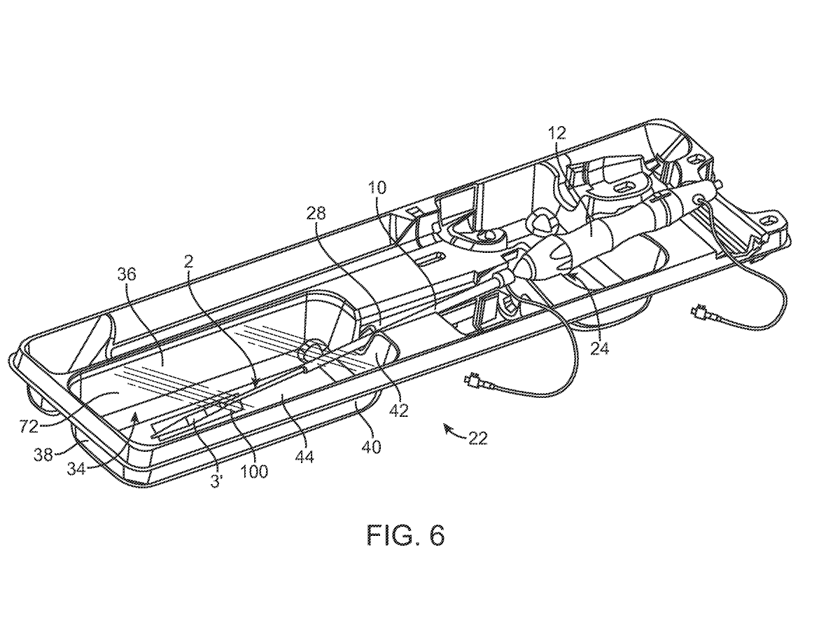

A method of loading a medical device on a catheter assembly using the loading tray and the temperature sensing and indicating devices described herein will now be described. The handle 12 of the catheter assembly 2 is seated in handle assembly receptacle 24 in tray body 23. Shaft assembly 10 of catheter assembly 2 is seated in elongate receptacle 28, and reservoir 34 is filled with a fluid 72, as shown in FIGS. 5-6. Either fluid 72 can be added to reservoir at the desired temperature or a temperature modifying agent is added to the fluid to reduce the fluid's temperature to the desired temperature. In an embodiment, fluid 72 is a saline solution and the temperature modifying agent (not shown) is a frozen saline solution. Accordingly, in such an embodiment, the saline solution and the frozen saline solution are added to reservoir 34. A user monitors temperature sensing and indicating device 100 to determine when the fluid reaches the desired temperature or temperature range. When the fluid 72 has reached the desired temperature or temperature range, the user loads the heart valve prosthesis 14 onto catheter assembly 2, as known to those skilled in the art. When catheter assembly is seated in tray 22 as described above, the distal tip 3 of catheter assembly 2 is suspended within reservoir 34. Distal tip 3 is below the horizontal plane defined by top surface 46 and above the bottom surface 44 of reservoir 34. Accordingly, when reservoir 34 is filled with the fluid 72, as illustrated in FIG. 6, distal tip 3 is submerged in fluid 72. Those skilled in the art would recognize that the fluid 72 can be added before or after catheter assembly is seated in the tray 22. However, the fluid 72 must be added and chilled to the desired temperature before the valve prosthesis 14 is loaded onto the catheter assembly 2.

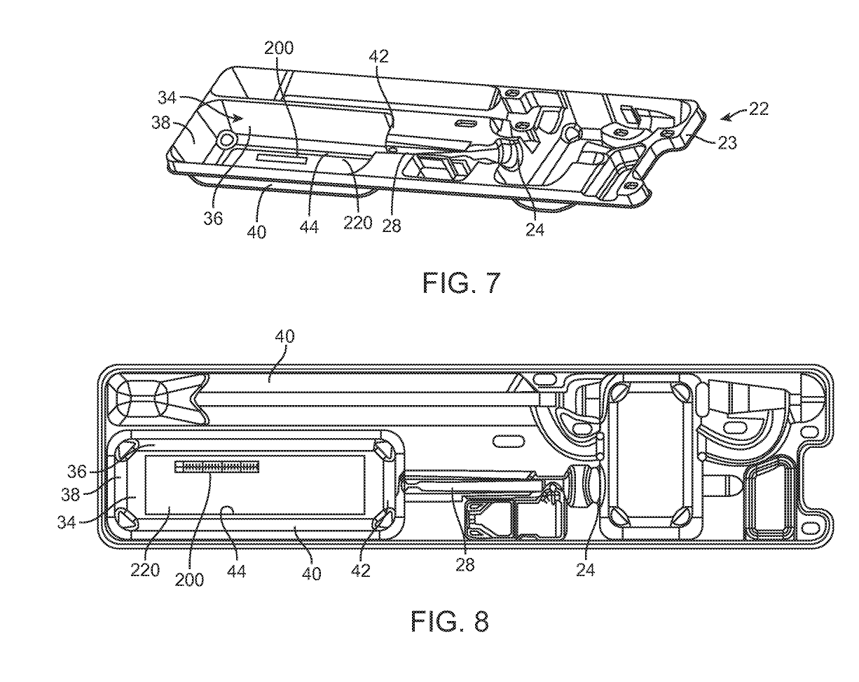

FIGS. 7-8 show another embodiment of a loading tray 22 including a visualization device or mirror 220 as described in co-pending U.S. application Ser. No. 13/658,082 filed Oct. 23, 2012, the disclosure of which is incorporated by reference herein in its entirety. Mirror 220 can be a conventional mirror or can be a mirror with magnification. As used herein, the term “mirror” means a reflecting surface such as, but not limited to, a polished metal or glass with a silvery, metallic, or amalgam backing. In one non-limiting example, mirror 220 has two-times magnification. Mirror 220 may be coupled to bottom surface 44 of reservoir by any means known to those skilled in the art. Mirror 220 may be a separate piece coupled to bottom surface 44, or can be formed integral with bottom surface 44. For example, and not by way of limitation, mirror 220 may be adhesively attached to bottom surface 44 such that mirror 220 faces the open top of reservoir 34. Mirror 220 may be coupled to bottom surface 44 of reservoir 34 in other ways such that a user utilizing tray 22 can see a reflection in the mirror 220 showing an underside of a catheter disposed within reservoir. For example, and not by way of limitation, mirror 220 may be snap fit into clips or other mechanism on bottom surface 44, or press fit onto bottom surface 44. As shown, mirror 220 covers substantially all of bottom surface 44 of reservoir 34. However, mirror 220 can be any suitable size such that mirror 220 can be used to observe a side of the catheter assembly facing mirror 220 when a portion of the catheter assembly is disposed in the reservoir. Further, the other embodiments described in U.S. application Ser. No. 13/658,082 filed Oct. 23, 2012 may be used with the temperature sensing and indicating devices described herein.

A temperature sensing and indicating device 200 is coupled to mirror 220. Temperature sensing and indicating device 200 may be similar to devices 100, 120 described above, or may be other temperature sensing and indicating devices known to those skilled in the art. In the embodiment shown, temperature sensing and indicating device 200 is coupled to mirror 220 by an adhesive. However, those skilled in the art would recognize that temperature sensing and indicating device 200 may be coupled to other surfaces of reservoir 34, may be coupled to mirror 220 by other mechanisms, mirror 220 may cover only a portion of bottom surface 44 and temperature sensing and indicating device 200 may be coupled to another portion, or other mechanisms for incorporating temperature sensing and indicating device 200 into reservoir may be used.

The method of a method of loading a medical device on a catheter assembly using the loading tray and the temperature sensing and indicating devices described herein as described with respect to FIGS. 5-6 applies equally to tray 22 with mirror 220 included therein. As described in U.S. application Ser. No. 13/658,082 filed Oct. 23, 2012, mirror 220 assisted the user in visualizing the portion of the catheter assembly facing bottom surface 44 such that connections required can be made visualized by the user without twisting the catheter assembly.

In another embodiment, a cooling mechanism, such as a mini-refrigeration unit, may be added to the tray to cool the fluid. In such an embodiment, a temperature sensing and indicating device is disposed in the reservoir to measure the temperature of the fluid therein, and to indicate when the temperature is at the desired temperature.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. It will also be understood that each feature of each embodiment discussed herein, and of each reference cited herein, can be used in combination with the features of any other embodiment. All patents and publications discussed herein are incorporated by reference herein in their entirety.

What is claimed is:

1. A tray for loading a medical device on a catheter assembly, the tray comprising:
a reservoir defined by a bottom surface, a first wall, a second wall, a third wall, and a fourth wall, the reservoir having a generally open top opposite the bottom surface, the reservoir configured to receive a liquid for loading the medical device on the catheter assembly; and
a temperature sensor disposed in the reservoir for measuring the temperature of the liquid and indicating when the liquid is at a desired temperature or temperature range for loading the medical device on the catheter assembly.

2. The tray of claim 1, wherein the temperature sensor is coupled to the bottom surface.

3. The tray of claim 2, wherein the temperature sensor is adhesively attached to the bottom surface.

4. The tray of claim 2, wherein the temperature sensor is coupled to the bottom surface using a snap fit.

5. The tray of claim 1, wherein the temperature sensor is coupled to one of the first wall, the second wall, the third wall, and the fourth wall.

6. The tray of claim 5, wherein the temperature sensor is adhesively attached to one of the first wall the second wall, the third wall, and the fourth wall.

7. The tray of claim 5, wherein the temperature sensor is coupled one of the first wall, the second wall, the third wall, and the fourth wall using a snap fit.

8. The tray of claim 1, further comprising a mirror coupled to the bottom surface of the reservoir, wherein the temperature sensor is coupled to the mirror.

9. The tray of claim 1, wherein the bottom surface of the reservoir includes a reflective surface, wherein the temperature sensor is coupled to the reflective surface.

10. The tray of claim 1, further comprising an indicator coupled to the temperature sensor for indicating when the liquid is at the desired temperature or temperature range for loading the medical device on the catheter assembly.

11. The tray of claim 10, wherein the indicator is a light.

12. The tray of claim 10, wherein the indicator is an audible alarm.

13. A method of loading a medical device on a catheter assembly comprising the steps of:
filling a reservoir in a loading tray with a fluid;
adding a temperature modifying agent to the fluid; and
waiting for a temperature sensor disposed in the reservoir to indicate that the fluid is at a desired temperature or temperature range.

14. The method according to claim 13 further comprising the step of loading a valve prosthesis on the distal tip of the catheter assembly while the valve prosthesis and the distal tip are submerged in the reservoir after the temperature of the fluid is at the desired temperature or temperature range.

15. The method according to claim 13, wherein the fluid is saline and the temperature modifying agent is chilled or frozen saline.

16. The method according to claim 13, wherein the reservoir includes a mirror coupled to a bottom surface of the reservoir, wherein the temperature sensor is coupled to the mirror.

17. The method according to claim 13, wherein the temperature sensor includes a temperature reading, and the step of waiting for the temperature sensor to indicate the temperature of the fluid comprises a user viewing the temperature reading.

18. The method according to claim 13, wherein the temperature sensor includes an indicator light coupled thereto which is activated when the temperature of the fluid reaches the desired temperature or temperature range.

19. The method according to claim 13, wherein the temperature sensor includes an audible alarm coupled thereto which is activated when the temperature of the fluid reaches the desired temperature or temperature range.
